(19) United States
(12) Patent Application Publication
(10) Pub. No.: US 2022/0339196 A1
(43) Pub. Date: Oct. 27, 2022

(54) PHARMACEUTICAL COMPOSITION FOR PREVENTING OR TREATING OBESITY OR NON-ALCOHOLIC FATTY LIVER, CONTAINING DENTAL TISSUE-DERIVED MULTIPOTENT STEM CELLS

(71) Applicants: INDUSTRY-ACADEMIC COOPERATION FOUNDATION GYEONGSNG NATIONAL UNVERSITY, Gyeongsangnam-do (KR); STEMCENTRIC CO., LTD., Gyeonggi-do (KR)

(72) Inventors: Gyu Jin Rho, Gyeongsangnam-do (KR); Kyung Min Kim, Seoul (KR); Si Jung Jang, Gyeongsangnam-do (KR); Sung Lim Lee, Gyeongsangnam-do (KR); Bong Wook Park, Gyeongsangnam-do (KR); Hyeon Jeong Lee, Gyeongsangnam-do (KR)

(21) Appl. No.: 17/640,725

(22) PCT Filed: Sep. 7, 2020
(86) PCT No.: PCT/KR2020/012045

§ 371 Date: Mar. 4, 2022

(30) Foreign Application Priority Data
Sep. 6, 2019 (KR) 10-2019-0110968

(51) Int. Cl. A61K 35/28; A61K 35/32; A61P 1/16; A61P 3/04

(52) U.S. Cl. CPC A61K 35/28; A61K 35/32; A61P 1/16; A61P 3/04

(57) ABSTRACT

The present invention relates to a pharmaceutical composition for preventing or treating obesity or non-alcoholic fatty liver, containing dental tissue-derived mesenchymal stem cells cryopreserved by vitrification. The pharmaceutical composition for preventing or treating obesity or non-alcoholic fatty liver, of the present invention, reduces body weight, lowers serum adipokine concentration, allows the liver to recover in view of histological findings, and improves lipid metabolic enzymes in the liver, thereby exhibiting remarkable prevention and treatment effects on obesity or non-alcoholic fatty liver. In addition, dental tissue-derived mesenchymal stem cells, which are the active ingredients of the present invention, are mesodermal stem cells having verified safety and may be obtained with the least invasiveness, and thus are expected to be effectively used as a cell therapeutic agent for obesity or non-alcoholic fatty liver.

### TECHNICAL FIELD

The present disclosure relates to a pharmaceutical composition for prevention or treatment of obesity or non-alcoholic fatty liver, the composition containing dental tissue-derived mesenchymal stem cells cryopreserved by vitrification.

### BACKGROUND ART

Obesity is a phenomenon in which excess energy is accumulated as fat in a body, resulting in abnormally high body fat level and thus, metabolic abnormalities. A cause thereof is estimated to include neuroendocrine causes, drug causes, decreased activity level, and genetic disease. Obesity is a chronic disease recognized as a cause of complications such as diabetes, heart disease, hypertension, and stroke. Treatment thereof mainly uses appetite suppressants and fat absorption inhibitors. Further, when implementing these obesity drug therapies, it is necessary to keep in mind risk of dependence, cardiovascular disease, and mood disorders in terms of side effects of obesity drugs. For example, sibutramine as an appetite suppressant has acted as a representative treatment agent in a domestic obesity treatment market, but a sale thereof has been discontinued due to possibility of increasing the risk of cardiovascular disease. Therefore, there is an urgent need to develop a therapeutic agent that has few side effects and may effectively control pathophysiology stages that cause the obesity.

Non-alcoholic fatty liver disease (NAFLD) is the most common chronic liver disease and is known to be closely related to type 2 diabetes mellitus, obesity, and metabolic syndrome. The number of non-alcoholic fatty liver disease patients is rapidly increasing in Korea due to westernized dietary habits, obesity, and increase in the diabetic population. According to a report from a GlobalData company, a NAFLD drug market is expected to reach approximately $25.3 billion by 2026. This is an average annual growth rate of 45%. However, exact pathogenesis of the non-alcoholic fatty liver disease has not been determined, and the number of subjects to study related to effects of drug treatment reported so far is not sufficient, and there is no officially approved treatment agent for the NAFLD. Therefore, there is a need for a new treatment agent that may secure stability during long-term use and may exert a distinct therapeutic effect on the NAFLD.

Further, dental tissue-derived mesenchymal stem cells are one type of mesenchymal stem cells. According to a recent study, the mesenchymal stem cells are found to be capable of differentiating into ectoderm (mainly, neurons) or endoderm (hepatocytes, pancreatic cells, etc.) as well as mesoderm.

### DISCLOSURE

### Technical Purpose

Thus, the present inventors have attempted to develop new therapeutic agents for obesity or non-alcoholic fatty liver. Then, we identified that after administration of dental tissue-derived mesenchymal stem cells cryopreserved by vitrification to a high-fat diet animal model, body weight loss, and decreased serum adipokine concentration were achieved, and the liver was recovered in terms of histological findings. Thus, it was confirmed that the dental tissue-derived mesenchymal stem cells were effective in treating the obesity or non-alcoholic fatty liver. In this way, the present disclosure has been completed.

Therefore, a purpose of the present disclosure is to provide a pharmaceutical composition for preventing or treating obesity or non-alcoholic fatty liver, the composition containing dental tissue-derived mesenchymal stem cells.

Another purpose of the present disclosure is to provide a stem cell therapeutic agent for treating obesity or non-alcoholic fatty liver, the agent containing dental tissue-derived mesenchymal stem cells.

### Technical Solution

In order to achieve the above purpose, the present disclosure provides a pharmaceutical composition for preventing or treating obesity or non-alcoholic fatty liver, the composition containing dental tissue-derived mesenchymal stem cells.

Further, the present disclosure provides a stem cell therapeutic agent for treating obesity or non-alcoholic fatty liver, the agent containing dental tissue-derived mesenchymal stem cells.

Further, the present disclosure provides a method for preventing or treating obesity or non-alcoholic fatty liver, the method comprising a step of treating a subject with dental tissue-derived mesenchymal stem cells.

Further, the present disclosure provides use of dental tissue-derived mesenchymal stem cells as a medicament for prevention or treatment of obesity or non-alcoholic fatty liver.

Further, the present disclosure provides use of dental tissue-derived mesenchymal stem cells to produce a pharmaceutical preparation for prevention or treatment of obesity or non-alcoholic fatty liver.

### Technical Effect

The pharmaceutical composition for the prevention or treatment of the obesity or non-alcoholic fatty liver according to the present disclosure may reduce the body weight, reduce the serum adipokine concentration, recover the liver in terms of histological findings, and improve lipid metabolism enzymes in the liver, and thus has significant prophylactic and therapeutic effects on the obesity or non-alcoholic fatty liver. Further, the dental tissue-derived mesenchymal stem cells as an active ingredient of the composition according to the present disclosure are mesenchymal stem cells having proven stability. The cells may be obtained in the most non-invasive way. Thus, the cells may be expected to be useful as a stem cell therapeutic agent for the obesity or non-alcoholic fatty liver.

### BRIEF DESCRIPTION OF DRAWINGS

FIG. 1 shows the results of measurement of body weights (A) and weights of liver tissue (B) of mice in each experimental group in which obesity and NAFLD are induced with a high-fat diet (*p<0.05, compared with control group; #p). <0.05, compared to HFD group).

FIG. 2 shows the results of histological analysis of the liver tissues of mice in the control group and each experimental group, wherein the H&E staining result in each row represents the histological findings of each of different mice (scale bar=50 μm, Magnification=×400).

FIG. 3 shows the Western blot results of liver tissues, and (A) shows the result of the expression level of androgen receptor (AR) in the liver tissue and (B) shows the result of quantifying the expression level (*p<0.05, compared to control group; #p<0.05, compared to HFD group).

FIG. 4 shows the results of ELISA analysis on the level of serum adipokine as an indicator of obesity (*p<0.05, compared with the control group; #p<0.05, compared with the HFD group).

### BEST MODE

The present disclosure provides a pharmaceutical composition for preventing or treating obesity or non-alcoholic fatty liver, the composition containing dental tissue-derived mesenchymal stem cells.

Hereinafter, the present disclosure will be described in detail.

Terms as used herein have meanings commonly used in the technical field to which the present disclosure belongs, unless otherwise defined herein.

In the present disclosure, the term “stem cells” is a generic term of undifferentiated cells in a stage before differentiation into each cell constituting the tissue. The stem cells may be differentiated into specific cells via specific differentiation stimulus (environment).

In the present disclosure, the term “mesenchymal stem cells (MSCs)” is also referred to as mesenchymal stem cells or adult stem cells, and refers to stem cells isolated from tissues other than embryos and then cultured. Mesenchymal stem cells exist in small amounts in most tissues that have already been differentiated, and presence thereof in almost all tissues studied so far, such as umbilical cord blood, umbilical cord, teeth, eyes, placenta, hair follicles, lungs, and liver has been confirmed.

In the present disclosure, the term “dental tissue-derived mesenchymal stem cells” refers to a concept including adult stem cells derived from tooth and dental tissue, mesenchymal stem cell-like cells having cell morphology similar to that of mesenchymal stem cells therefrom. Compared to other adult stem cells present in bone marrow or umbilical cord, the dental tissue-derived mesenchymal stem cells may be easily obtained while the tissue damage is minimized (in a non-invasive manner). Thus, the dental tissue-derived mesenchymal stem cells are used as an active ingredient of the composition in accordance with the present disclosure. The dental tissue may be dental pulp tissue of deciduous teeth or permanent teeth, periodontal ligament tissue, dental follicle tissue of erupting teeth, tooth germ stem tissue, and apical papilla tissue of immature permanent teeth, and may be preferably dental tissue of wisdom teeth. However, the disclosure is not limited thereto.

Further, the tissue may be characterized in that the tissue includes mesenchymal stem cells, wherein the mesenchymal stem cells may be one or more selected from a group consisting of dental pulp stem cells (DPSCs), stem cells from human exfoliated deciduous teeth (SHEDs), periodontal ligament stem cells (PDLSCs), dental follicle stem cells (DFSCs), and stem cells from apical papilla (SCAP). However, the disclosure is limited thereto.

In one example, the dental tissue-derived mesenchymal stem cells have been clinically applied to regenerative medicine to treat or reduce peri-implantitis, nerve damage, and loss of gum bone. However, so far, there has been no report that the dental tissue-derived mesenchymal stem cells have the medical effect on obesity or non-alcoholic liver disease.

In the present disclosure, the dental tissue is characterized in that it is separated from the human body and is cryopreserved by vitrification. The cryopreservation method by the vitrification may use a method described in Korean Patent No. 10-1551900 as a prior patent of the present inventors.

In the present disclosure, a method for isolating mesenchymal stem cells include a method known in the art without limitation. For example, the mesenchymal stem cells may be isolated from the dental tissue and purified, and the separated mesenchymal stem cells may be cultured as needed.

In the present disclosure, the obesity means a body fat index of 25 kg/m² or higher as the obesity reference presented by the Korean Society of Obesity. Thus, the obesity may mean various metabolic syndromes and cardiovascular diseases including arteriosclerosis, type 2 diabetes, and myocardial infarction due to the obesity. The disclosure is not limited thereto.

Further, the non-alcoholic fatty liver disease (NAFLD) refers to a case in which the fat content in liver cells not caused by alcohol is 5% or greater of a liver weight. The NAFLD refers to a state in which simple accumulation of fat (triglycerides, fatty acids, etc.) in the liver tissue has occurred, a hepatic steatosis state, or a disease state in which an inflammatory reaction in the liver tissue as a result of an increase in fat accumulation in the liver tissue has occurred. However, the disclosure is not limited thereto.

In the present disclosure, “a pharmaceutical composition for prevention or treatment of obesity or non-alcoholic fatty liver, the composition containing dental tissue-derived mesenchymal stem cells” may be dental tissue-derived mesenchymal stem cells themselves, or may refer to composition containing the dental tissue-derived mesenchymal stem cells, a culture medium, or a substance commonly used in the art when culturing stem cells. Further, a substance secreted from the dental tissue-derived mesenchymal stem cells or a substance extracted from the dental tissue-derived mesenchymal stem cells may be used as a substitute for the dental tissue-derived mesenchymal stem cells.

In the present disclosure, the pharmaceutical composition may contain a conventional pharmaceutically acceptable carrier in addition to the dental tissue-derived mesenchymal stem cells. An injection may contain a preservative, an analgesic agent, a solubilizer or stabilizer as the carrier. A formulation for topical administration may contain a base, an excipient, a lubricant or a preservative as the carrier.

The pharmaceutically acceptable carriers contained in the pharmaceutical composition according to the present disclosure are commonly used in formulations, and may include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate and mineral oil. The disclosure is limited thereto. The pharmaceutical composition according to the present disclosure may further contain lubricants, wetting agents, sweetening agents, flavoring agents, emulsifying agents, suspending agents, preservatives, etc. in addition to the above ingredients.

The pharmaceutical composition according to the present disclosure may be formulated into a unit dosage form or may be incorporated into a multi-dose container, using a pharmaceutically acceptable carrier and/or excipient, according to a method that may be easily performed by a person of ordinary skill in the art to which the present disclosure belongs. In this case, the formulation may be in the form of a solution, suspension or emulsion in an oil or aqueous medium, and may additionally contain a dispersant or stabilizer.

The pharmaceutical composition according to the present disclosure may be administered parenterally, and may be administered intravenously, subcutaneously, intraperitoneally or topically. The composition for parenteral administration (e.g., injection) of the composition according to the present disclosure may be dispersed and/or dissolved in a pharmaceutically acceptable carrier, for example, sterile purified water, a buffer of about pH 7, or physiological saline and then may be injected into the body. If necessary, the composition may contain conventional additives such as preservatives, stabilizers, and the like.

Further, in the present disclosure, the mesenchymal stem cells may be administered at an amount from 10⁴ to 10¹⁰ cells/one time, preferably from 10⁵ to 10⁹ cells/one time, more preferably, from 10⁶ to 10⁸ cells/one time, most preferably, of 5×10⁷ to 10⁸ cells/one time. However, the disclosure is not limited thereto. The dosage may be prescribed in various ways depending on factors such as formulation method, administration mode, age, weight, pathology, food, administration time, administration route, excretion rate, and response sensitivity of the patient.

In the present disclosure, the pharmaceutical composition may be characterized in that the composition may induce weight loss, reduction of blood adipokine concentration, and histological recovery of the liver to reduce the obesity or non-alcoholic fatty liver caused by high-fat diet intake or metabolic syndrome.

In one embodiment of the present disclosure, it is confirmed that when the dental tissue-derived mesenchymal stem cells are administered to the high-fat diet animal model, the weight of the high-fat diet animal model is reduced, and the fat deposition in the liver tissue is significantly reduced, the serum adipokines, TNF-α and IL-1β were decreased, thereby reducing the obesity and non-alcoholic fatty liver. In this connection, the serum adipokine is secreted from adipocytes activated in an obesity state, and is composed of proinflammatory cytokines including TNF-α and IL-1β. It has been reported that in the case of the non-alcoholic fatty liver, the liver exhibits pathological symptoms locally similar to obesity. Thus, based on the above results, it may be identified that the dental tissue-derived mesenchymal stem cells according to the present disclosure lowers the adipokine concentration via the activation of fat metabolism, thereby preventing or treating the obesity or non-alcoholic fatty liver.

The pharmaceutical composition according to the present disclosure may be used alone, or in combination with methods for preventing or treating obesity or non-alcoholic fatty liver known in the art, for preventing and treating obesity or non-alcoholic fatty liver.

Further, the present disclosure provides a stem cell therapeutic agent for treating obesity or non-alcoholic fatty liver, the agent containing dental tissue-derived mesenchymal stem cells.

In the present disclosure, the dental tissue may be dental pulp tissue of deciduous teeth or permanent teeth, periodontal ligament tissue, dental follicle tissue of erupting teeth, tooth germ stem tissue, and apical papilla tissue of immature permanent teeth, and may be preferably dental tissue of wisdom teeth. However, the disclosure is not limited thereto.

In the present disclosure, the term “cell therapeutic agent” refers to cells and tissues produced via isolation, culturing, and special manipulation from humans, and acting as pharmaceutical products (US FDA regulations) used for the purpose of treatment, diagnosis, and prophylaxis. The term “cell therapeutic agent” refers to medicines that are used for the purpose of treatment, diagnosis and prevention via a series of actions such as proliferating or selecting living autologous, allogeneic or xenogeneic cells in vitro or changing the biological characteristics of cells in other ways in order to restore the function of cells or tissues.

The term “prevention” used in the present disclosure refers to any action that suppresses obesity or non-alcoholic fatty liver or delays the progression thereof via administration of the pharmaceutical composition or stem cell therapeutic agent according to the present disclosure.

The term “treatment” used in the present disclosure refers to any action by which the obesity or non-alcoholic fatty liver is reduced or changed to a beneficial effect via administration of the pharmaceutical composition or stem cell therapeutic agent according to the present disclosure.

Further, the present disclosure provides a method for preventing or treating obesity or non-alcoholic fatty liver, the method comprising a step of treating a subject with the dental tissue-derived mesenchymal stem cells.

Further, the present disclosure provides use of the dental tissue-derived mesenchymal stem cells as a medicament for the prevention or treatment of obesity or non-alcoholic fatty liver.

Further, the present disclosure provides use of the dental tissue-derived mesenchymal stem cells to produce a pharmaceutical preparation for the prevention or treatment of obesity or non-alcoholic fatty liver.

Hereinafter, the present disclosure will be described in more detail based on Examples. These Examples are only for describing the present disclosure in more detail. It will be apparent to those of ordinary skill in the art that the scope of the present disclosure is not limited to these Examples, according to the gist of the present disclosure. Statistical analysis was performed according to one-way ANOVA followed by Tukey's test using SPSS (SPSS Inc., Chicago, Ill., USA). Results are presented as mean±standard error. P<0.05 was determined to be significant.

### EXAMPLES

### Example 1. Preparation of Dental Tissue-Derived Mesenchymal Stem Cells

All chemical substances were purchased from SigmaAldrich® (St. Louis, Mo., USA), and the medium was purchased from Gibco Life Technologies (Gaithersburg, Md., USA). To obtain the dental tissue-derived mesenchymal stem cells, the present inventors established a new cryopreservation protocol which was named a cryopreservation method by vitrification. The method is configured for effectively preserving cells in tissues using a cryopreserving composition by vitrification of tissues containing ethylene glycol, sucrose and glucose, and is described in Korean Patent No. 10-1551900, which is a prior patent of the present inventors, and is incorporated herein by reference in its entirety.

More specifically, a dental tissue that was extracted from an impacted wisdom teeth patient around the age of 20 and then discarded was provided from the Department of Oral and Maxillofacial Surgery at Gyeongsang National University Hospital, and the tooth tissue was separated from the extracted wisdom tooth using a sterile scalpel and was frozen by vitrification. and stored for at least 3 months before thawing. When thawing the tissue, a freezing tube stored in liquid nitrogen was thawed in water at 37° C. for 1 to 2 minutes. In order to completely remove a cryopreserving agent, centrifugation at 1500 rpm was performed for 5 minutes using DPBS containing 100 U/ml penicillin/streptomycin and 0.25 μg/ml amphotericin B (amphotericin B, Invitrogen) and then washing was performed several times. In order to isolate mesenchymal stem cells from the dental tissue, the tissue was enzymatically treated with collagenase type IV (Sigma, USA) for 30 minutes. After filtering the treated tissue once through each of a 100 μm cell strainer (BD Falcon™, USA) and a 40 μm cell strainer (BD Falcon™, USA), we transferred the tissue to a 15 ml polypropylene conical tube (BD Falcon™, USA) and then centrifuged the tissue at 1500 rpm for 5 minutes using DPBS containing penicillin/streptomycin and amphotericin B (amphotericin B, Gibco, USA) and then washed the tissue two times.

The mesenchymal stem cells derived from the dental tissue as cryopreserved by vitrification were cultured using 4 mL DMEM (Dulbecco's modified Eagle's Medium) containing 10% fetal bovine serum (FBS), 1% L-glutamine (Glutamax™ and 1% penicillin/streptomycin and in 25 T-flasks (Nunc™, Roskilde, Denmark) and under 5% CO₂, 37° C. and humid conditions. The medium was replaced once every 3 days Immediately before administration of the cells to animals, the cells subcultured 4 to 5 times were removed from a bottom of the culture vessel with 0.25% trypsin/EDTA and were washed with DPBS. The separated cells were counted using a hemocytometer, and 1×10⁶ cells were diluted in 200 μl of cold DPBS and were used for subsequent experiments (hereinafter referred to as MSCs).

### Example 2. Preparation of Animal Model Via High-Fat Diet Intake

Forty 8-week-old male C57BL/6J mice weighing 20 to 22 g were obtained from Central Lab Animal Inc. (Seoul, Korea). All mice were raised under a 12-hour light-dark cycle, room temperature 25±2° C., and humidity 30 to 40%, and were freely accessible to water and food. Mice were randomly divided into 4 groups:

1. Control group (Con) (n=10): normal diet for 10 weeks

2. HFD group (n=10): high fat diet including 60 kcal % fat for 10 weeks+200 μl PBS intraperitoneal administration after 10 weeks

3. MSCi group (n=10): high fat diet including 60 kcal % fat for 10 weeks+1×10⁶/200 μl MSCs intraperitoneally administration after each of 5 weeks, 7 weeks and 9 weeks of high-fat diet

4. MSCs group (n=10): high fat diet including 60 kcal % fat for 10 weeks+1×10⁶/200 μl MSCs intraperitoneally administration for 5 consecutive days after 8 weeks of high-fat diet

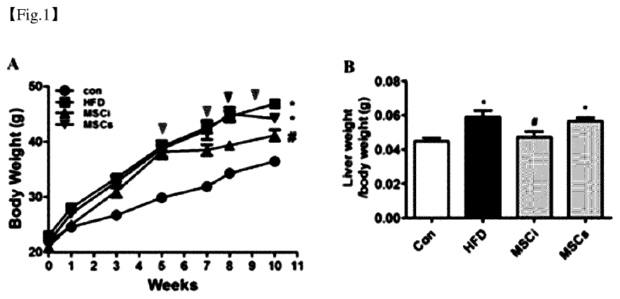

All mice were weighed weekly, and after 10 weeks, 0.5 μL/g tiletamine-zolazepam (Zoletil® and 0.5 μL/g xylazine (Rompun, Bayer Korea Ltd., Seoul, Korea) were injected thereto and the mice were sacrificed. The mouse blood was collected via cardiac puncture, and a residual blood was removed with perfusion with PBS containing heparin. Liver tissue was obtained and weighed for tissue weight/body weight calculation. All animal experiments were performed according to the guidelines for the treatment and use of laboratory animals of Gyeongsang National University. The results are shown in FIG. 1.

As shown in A of FIG. 1, the body weight of the HDF group was significantly higher than that of the control group from the 3rd week of the high-fat diet (p<0.05). The body weight of the MSCi group was significantly lower (p<0.05) compared to the HFD group at the time of the first administration of MSC. In the case of liver weight, as shown in B of FIG. 1, the liver weight of the HFD group was significantly increased compared to that of the control group (p<0.05). The MSCi group exhibited a significantly reduced liver weight compared to the HFD group (p<0.05), whereas the MSCs group exhibited no significant difference compared to the HFD group.

### Example 3. Histological Analysis Before and After MSC Administration

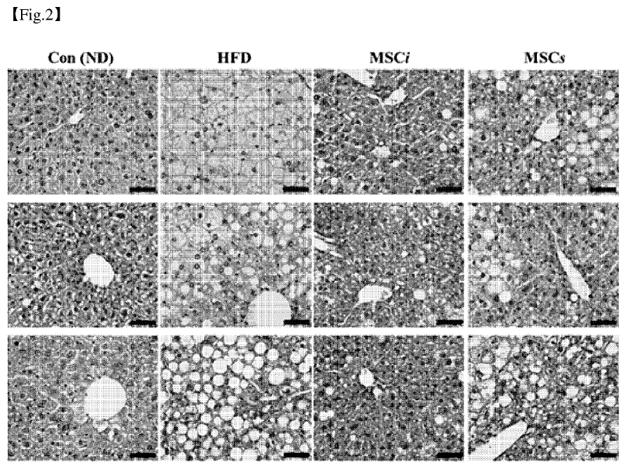

To identify the therapeutic effect of the dental tissue-derived mesenchymal stem cells on the liver, histological analysis was performed. Liver tissue was fixed in 4% formaldehyde and dehydrated, then embedded in paraffin and cut into 5 μm thick sections. After removing the paraffin component from the tissue section using xylene, rehydration with ethanol was performed, and then staining with hematoxylin and eosin (H&E) was performed. All stained tissues were dehydrated and washed, and mounted on a permount (Fisher scientific, NH, USA), and then an amount of staining was identified with an optical microscope (Nikon Eclipse 80i) and Photo Imaging System (Canon 600D). The results are shown in FIG. 2.

As shown in FIG. 2, it was identified based on a result of histological analysis of the liver tissue that the control group exhibited characteristics of normal liver tissue, while the HFD group exhibited macrovesicular steatosis and microvesicular steatosis as a serious symptom. Microvesicular steatosis is a histological feature of NAFLD. Further, it was identified that the liver tissue which exhibited severe fat deposition in both MSCs and MSCi groups was recovered to a level similar to that of the normal model after the stem cell administration thereto, and thus, hepatic steatosis was reduced. The reduction thereof was greater in the MSCi group than that in the MSCs group.

### Example 4. Assessment of Lipid Homeostasis Due to MSC Administration

4-1. Western Blot

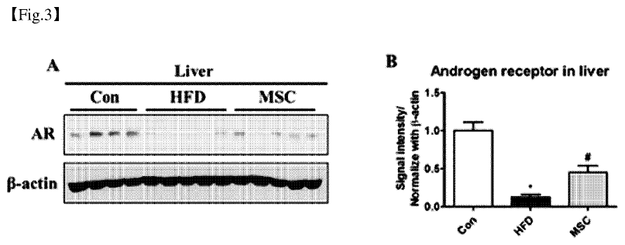

In order to identify the effect of administration of dental tissue-derived mesenchymal stem cells on lipid homeostasis, western blot was performed on liver tissue, and an expression level of androgen receptor (AR) which induces lipolysis via lipid oxidation in liver and adipose tissue was analyzed. Liver tissues were collected from each experimental group and stored at −80° C. until use. Each tissue was lysed using RIPA buffer (PIERCE, Rockford, Ill., USA) to extract a protein. Then, the extracted protein was quantified using the BCA protein analysis kit (PIERCE). 50 μg of protein was loaded on SDS-PAGE which was transferred to a nitrocellulose membrane (Amersham Pharmacia Biotech, Piscataway, N.J., USA). The membrane reacted with primary monoclonal anti-androgen receptor (ab133273, Abcam) diluted at 1:1000, and beta-actin (a5441, sigma) diluted at 1:5000 overnight at 4° C. After washing 3 times with 0.1% TBST, HRP-conjugated goat anti-mouse (Invitrogen) and goat anti-rabbit (Invitrogen) secondary antibodies diluted at 1:10000 were added thereto and then reaction occurred for 1 hour at room temperature. The membrane was developed using an ECL kit (Amersham Pharmacia Biotech, Piscataway, N.J., USA), and the optical density of the target protein relative to beta-actin was measured using ImageJ software (NIH, USA). The results are shown in FIG. 3.

As shown in FIG. 3, it was identified that the concentration of AR in the liver was significantly decreased (p<0.05), but significantly increased after MSC administration. Based on the above results, it was identified that lipid metabolism including lipid oxidation was increased in liver tissue via AR recovery.

4-2. ELISA

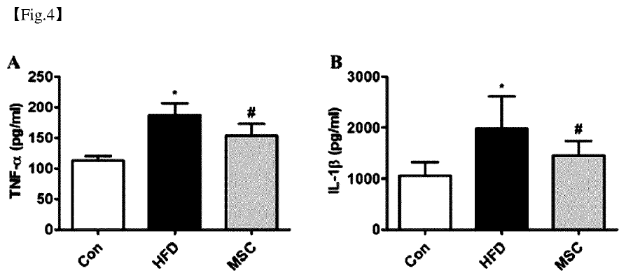

In order to identify the relationship between weight loss and recovery of AR expression, the level of serum adipokine which is an obesity index was measured using an ELISA method. Mouse blood was collected via cardiac puncture as described in above Example 2, and serum was collected via centrifugation at 400×G for 10 minutes and was stored at −80° C. until use. To identify whether the dental tissue-derived mesenchymal stem cells may reduce the obesity and non-alcoholic fatty liver induced via high-fat diet, TNF-α (ADI-900-047, ENZO) and IL-1β in the mouse serum was analyzed via ELISA. The results are shown in FIG. 4.

As shown in FIG. 4, both TNF-α and IL-1β which are serum adipokines were significantly increased in the HFD group (p<0.05), but decreased after MSC administration. Based on the above results, it was identified that administration of dental tissue-derived mesenchymal stem cells could reduce the inflammatory state in obesity and thus reduce the obesity.

It is reported that among the various animal models having the induced NAFLD, the high-fat diet animal model has the pathological features most similar to those of the human NAFLD, and exhibits similar physiological changes such as weight gain, increased blood sugar, and increased metabolic disease.

The present inventors have prepared an animal model induced via a high-fat diet to develop novel therapeutic agents for obesity and NAFLD. It was identified that after administration of the dental tissue-derived mesenchymal stem cells to the animal model, obesity and NAFLD were reduced via weight loss, normalization of blood adipokine levels, and improvement of liver lipid metabolizing enzymes.

In addition, the dental tissue-derived mesenchymal stem cells as the active ingredient of the composition according to the present disclosure may be obtained in the most non-invasive way, and thus is expected to be useful as a cell treatment agent for obesity or non-alcoholic fatty liver.

The present disclosure has been described based on the specific embodiments in detail. Those of ordinary skill in the art will appreciate that the specific embodiments are only preferred implementation examples. It is clear that the scope of the present disclosure is not limited thereto. Accordingly, a substantial scope of the present disclosure will be defined by the appended claims and their equivalents.

1.-9. (canceled)

10. A method for preventing or treating obesity or non-alcoholic fatty liver, the method comprising administering dental tissue-derived mesenchymal stem cells to a subject.

11. The method of claim 10, wherein the dental tissue includes at least one selected from a group consisting of a dental pulp tissue of a deciduous tooth or a permanent tooth, a periodontal ligament tissue, a dental follicle tissue, a tooth germ stem tissue, and an apical papilla tissue.

12. The method of claim 10, wherein the dental tissue includes a dental tissue of a wisdom tooth.

13. The method of claim 10, wherein the dental tissue is cryopreserved by vitrification.

14. A stem cell therapeutic agent for treating obesity or non-alcoholic fatty liver, the agent containing dental tissue-derived mesenchymal stem cells.

15. The stem cell therapeutic agent of claim 14, wherein the dental tissue includes at least one selected from a group consisting of a dental pulp tissue of a deciduous tooth or a permanent tooth, a periodontal ligament tissue, a dental follicle tissue, a tooth germ stem tissue, and an apical papilla tissue.

16. Use of dental tissue-derived mesenchymal stem cells as a medicine for prevention or treatment of obesity or non-alcoholic fatty liver.

17. Use of dental tissue-derived mesenchymal stem cells to produce a pharmaceutical preparation for prevention or treatment of obesity or non-alcoholic fatty liver.
